(19) United States
(12) Patent Application Publication
(10) Pub. No.: US 2026/0182933 A1
(43) Pub. Date: Jul. 2, 2026

(54) Customized Alerts for Low Blood Glucose Levels

(71) Applicants: Hana Hafezzadeh, San Jose, CA (US); Bejan Hafezzadeh, San Jose, CA (US)

(72) Inventors: Hana Hafezzadeh, San Jose, CA (US); Bejan Hafezzadeh, San Jose, CA (US)

(21) Appl. No.: 19/006,875

(22) Filed: Dec. 31, 2024

(51) Int. Cl. A61B 5/00; A61B 5/145

(52) U.S. Cl. CPC A61B 5/746; A61B 5/14532; A61B 5/4809; A61B 5/7405; A61B 5/7455

(57) ABSTRACT

In accordance with some implementations, a method includes obtaining blood glucose (BG) data indicative of BG levels of an individual. The method includes obtaining a first sleepiness score associated with the individual. The method includes determining, based at least in part on the BG data, that a low blood sugar alert condition is satisfied. The method includes, in response to determining that the low blood sugar alert condition is satisfied, directing an alert subsystem to generate a first alert output based on the first sleepiness score.

### TECHNICAL FIELD

The present disclosure relates to systems, methods, and devices of providing alerts of low blood glucose (BG) levels.

### BACKGROUND

A blood glucose (BG) system may provide an alert to an individual when the individual experiences low BG levels. However, when the individual is sleeping or unconscious, the alert may be inadequate to wake the individual, potentially resulting in hypoglycemia. Moreover, the BG system does not include a mechanism for providing customized alerts to the individual.

### SUMMARY

In accordance with some implementations, a method includes obtaining BG (BG) data indicative of BG levels of an individual. The method includes obtaining a first sleepiness score associated with the individual. The method includes, determining, based at least in part on the BG levels, that a low blood sugar alert condition is satisfied. The method includes, in response to determining that the low blood sugar alert condition is satisfied, directing an alert subsystem to generate a first alert output based on the first sleepiness score.

In accordance with some implementations, a method is performed at an electronic device including one or more processors and a non-transitory memory. The method includes obtaining BG data indicative of BG levels of an individual. The method includes obtaining a first sleepiness score associated with the individual. The method includes, determining, based at least in part on the BG levels, that a low blood sugar alert condition is satisfied. The method includes, in response to determining that the low blood sugar alert condition is satisfied, directing an alert subsystem to generate a first alert output based on the first sleepiness score.

In accordance with some implementations, an electronic device includes one or more processors and a non-transitory memory. One or more programs are stored in the non-transitory memory and are configured to be executed by the one or more processors. The one or more programs include instructions for performing or causing performance of the operations of any of the methods described herein. In accordance with some implementations, a non-transitory computer readable storage medium has stored therein instructions which when executed by one or more processors of an electronic device, cause the device to perform or cause performance of the operations of any of the methods described herein. In accordance with some implementations, an electronic device includes means for performing or causing performance of the operations of any of the methods described herein. In accordance with some implementations, an information processing apparatus, for use in an electronic device, includes means for performing or causing performance of the operations of any of the methods described herein.

### BRIEF DESCRIPTION OF THE DRAWINGS

For a better understanding of the various described implementations, reference should be made to the Description, below, in conjunction with the following drawings in which like reference numerals refer to corresponding parts throughout the figures.

FIG. 1 is an example of an environment in accordance with some implementations.

FIG. 2 is an example of a block diagram of a sleepiness score generator in accordance with some implementations.

FIG. 3 is an example of a first timing diagram and a second timing diagram in accordance with some implementations.

FIG. 4 is an example of a flow diagram of a method of providing customized alerts according to various implementations.

### DESCRIPTION OF IMPLEMENTATIONS

A BG system may provide an alert to an individual when the individual experiences low BG levels. For example, the BG system may direct an audio output device (e.g., a speaker) to play audio, in an attempt to wake an individual. However, played audio often fails to wake certain sleepers, potentially resulting in hypoglycemia. Moreover, the BG system does not include a mechanism for providing customized alerts to an individual. For example, an individual may manually select an alert type (e.g., audio or vibration), but the BG system does not automatically provide different alert types for different situations.

By contrast, various implementations disclosed herein include methods, electronic devices, and BG systems for providing customized alerts to an individual experiencing low blood sugar levels. To that end, in some implementations, a method includes obtaining a sleepiness score associated with the individual, and selecting an alert type of a plurality of alert types, based on the sleepiness score. Moreover, in some implementations, the method includes directing a device to generate an alert output based on the selected alert type. In some implementations, the sleepiness score indicates a confidence that the individual is asleep. In some implementations, the sleepiness score indicates how deeply the individual is sleeping—e.g., a high sleepiness score indicates deep sleep, whereas a moderate sleepiness score indicates rapid eye movement (REM) sleep. For example, the method includes selecting an audio alert based on a relatively low sleepiness score, and selecting a haptic alert based on a relatively high sleepiness score. As another example, the method includes directing an audio output device (e.g., a speaker) to play a tone at a first volume level based on a relatively low sleepiness score, and directing the audio output device to play the tone at a second volume level based on a relatively high sleepiness score, wherein the second volume level is higher than the first volume level. In some implementations, the BG system may be integrated in a continuous glucose monitoring (CGM) device and/or integrated in an insulin pump.

In various implementations, the method includes determining the sleepiness score based on one or more of biometric data associated with the individual and environmental data characterizing an environment associated with the individual. The biometric data may indicate heart rate information associated with the individual, breathing rate information associated with the individual, blood oxygen information associated with the individual, etc. The environmental data may indicate ambient light information regarding the environment (e.g., captured by an ambient light sensor), and may include image data regarding the environment. For example, the image data represents one or images of the environment, wherein the images may be captured by a camera being worn by the individual (e.g., a camera integrated into a CGM or pump). The method may include performing computer vision with respect to the image data to identify certain characteristics of the environment that indicate the individual is sleeping. For example, the method includes determining a relatively high sleeping score, based on determining a majority of pixel values of the image data indicate a dark color (e.g., black).

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described implementations. The first contact and the second contact are both contacts, but they are not the same contact, unless the context clearly indicates otherwise.

The terminology used in the description of the various described implementations herein is for the purpose of describing particular implementations only and is not intended to be limiting. As used in the description of the various described implementations and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes”, “including”, “comprises”, and/or “comprising”, when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting”, depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]”, depending on the context.

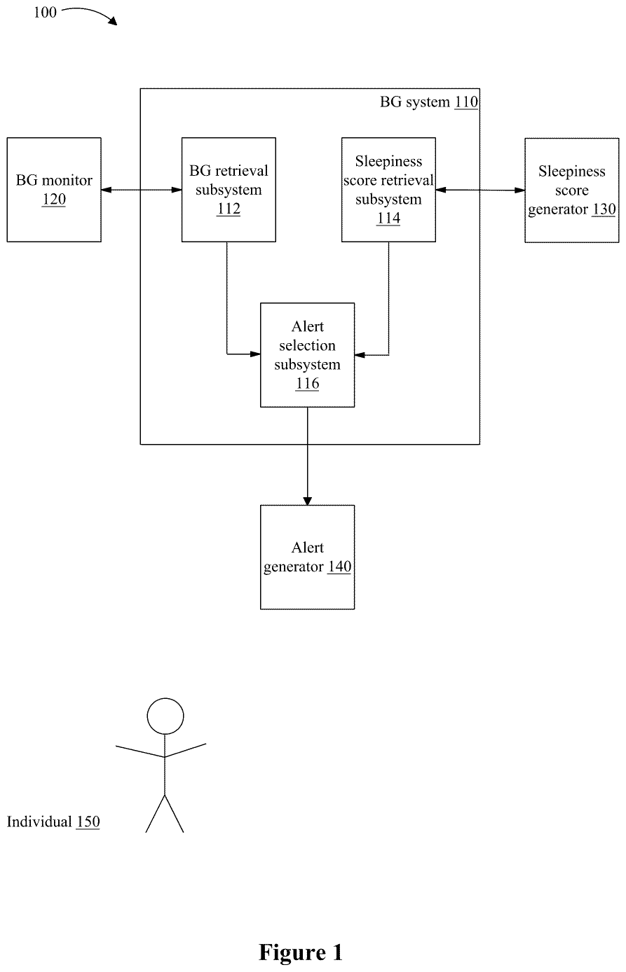

FIG. 1 is an example of an environment 100 in accordance with some implementations. For example, the environment 100 corresponds to a physical environment including an individual 150, such as the bedroom of the individual 150. The environment 100 includes a BG (BG) system 110, a BG monitor 120, a sleepiness score generator 130, and an alert generator 140. Although each of the BG monitor 120, the sleepiness score generator 130, and the alert generator 140 is illustrated are being separate from the BG system 110, in some implementations some or all of the BG monitor 120, the sleepiness score generator 130, and the alert generator 140 are integrated in the BG system 110.

The BG monitor 120 monitors BG levels of the individual 150 and generates BG data indicative of the BG levels (e.g., BG levels in units of milligrams per deciliter (mg/dL). For example, in some implementations, the BG monitor 120 corresponds to or is integrated in a continuous glucose monitor (CGM) device. The BG data may indicate a plurality of BG levels of the individual 150 at a corresponding plurality of times. For example, a first portion of the BG data indicates that at a first time the individual 150 has a 75 BG level, a second portion of the BG data indicates that a second time the individual 150 has a 70 BG level, a third portion of the BG data indicates that a third time the individual 150 has a 50 BG level, etc.

The BG system 110 includes a BG retrieval subsystem 112, a sleepiness score retrieval subsystem 114, and an alert selection subsystem 116. In some implementations, the BG system 110 includes a controller and a non-transitory memory (e.g., random access memory (RAM)). For example, the controller corresponds to one or more processors (e.g., one or more central processing units (CPUs)). Moreover, the non-transitory memory may include instructions which, when executed by the controller, causes the controller to perform respective operations of one or more the BG retrieval subsystem 112, the sleepiness score retrieval subsystem 114, and the alert selection subsystem 116. The respective operations of the BG retrieval subsystem 112, the sleepiness score retrieval subsystem 114, and the alert selection subsystem 116 are described below.

The BG retrieval subsystem 112 obtains, from the BG monitor 120, the BG data indicative of BG levels of the individual 150. To that end, in some implementations, the BG retrieval subsystem 112 is communicatively coupled with the BG monitor 120, enabling the BG retrieval subsystem 112 to receive the BG data from the BG monitor 120. For example, the BG retrieval subsystem 112 and the BG monitor 120 communicate with each other via Bluetooth. In some implementations, the BG monitor 120 is integrated in the BG system 110. For example, each of the BG monitor 120 and the BG system 110 is integrated in a CGM device worn by the individual 150. In some implementations, the BG monitor 120 persistently transmits BG data to the BG retrieval subsystem 112—e.g., the BG monitor 120 transmits portions of the BG data periodically to the BG retrieval subsystem 112. In some implementations, the BG monitor 120 transmits portions of the BG data to the subsystem 112 in response to receiving corresponding request(s) from the BG retrieval subsystem 112.

The sleepiness score retrieval subsystem 114 obtains, from the sleepiness score generator 130, one or more sleepiness scores associated with the individual 150. To that end, in some implementations, the sleepiness score retrieval subsystem 114 is communicatively coupled with the sleepiness score generator 130, enabling the sleepiness score retrieval subsystem 114 to receive the sleepiness score(s) from the sleepiness score generator 130. For example, the sleepiness score retrieval subsystem 114 and the sleepiness score generator 130 communicate with each other via Bluetooth. As one example, the sleepiness score generator 130 is integrated in a smartwatch being worn by the individual 150, and the smartwatch may monitor biometric(s) of the individual 150 in order to generate a sleepiness score. In some implementations, the sleepiness score generator 130 is integrated in the BG system 110. In some implementations, the sleepiness score generator 130 persistently transmits the sleepiness score(s) to the sleepiness score retrieval subsystem 114—e.g., the sleepiness score generator 130 transmits the sleepiness score(s) periodically to the sleepiness score retrieval subsystem 114. In some implementations, the sleepiness score generator 130 transmits a sleepiness score to the sleepiness score retrieval subsystem 114 in response to receiving corresponding request from the sleepiness score retrieval subsystem 114.

A sleepiness score may indicate a (e.g., current) sleeping status of the individual 150. In some implementations, a sleepiness score indicates a confidence that the individual 150 is asleep. For example, the sleepiness score may range from 0.0 to 1.0, wherein a higher sleepiness score indicates a higher confidence that a user is sleeping. In some implementations, a sleepiness score indicates a sleep stage associated with the individual 150. For example, a sleepiness score may be a first value for wake sleep stage, a second value for a nonrapid eye movement (NREM) sleep stage, and a third value for rapid eye moment (REM) sleep stage. As another example, a sleepiness score may be a first value for N1 sleep stage, a second value for N2 sleep stage, a third value for N3 sleep stage, and a fourth value for REM sleep stage. In some implementations, a sleepiness score indicates both a sleep stage and a confidence associated with the individual 150 sleeping according to the sleep stage. In some implementations, the sleepiness score retrieval subsystem 114 obtains, from the sleepiness score generator 130, a plurality of sleepiness scores associated with a corresponding plurality of times. For example, a first sleepiness score indicates a confidence that the individual 150 is asleep at a first time, a second sleepiness score indicates a confidence that the individual 150 is asleep at a second time, a third sleepiness score indicates a confidence that the individual 150 is asleep at a third time, etc.

The alert selection subsystem 116 obtains, from the BG retrieval subsystem 112, the BG data indicative of BG levels of the individual 150, and obtains, from the sleepiness score retrieval subsystem 114, the sleepiness score(s). Based on the BG levels and the sleepiness score(s), the alert selection subsystem 116 selects a particular alert type of a plurality of alert types, and directs the alert generator 140 to generate an alert output corresponding to the particular alert type. To that end, in some implementations, alert selection subsystem 116 transmits, to the alert generator 140, an indication of the particular alert type. In some implementations, the alert selection subsystem 116 transmits and the alert generator 140 are communicatively coupled to each other (e.g., via Bluetooth or Wi-fi). In some implementations, the alert generator 140 is integrated in the BG system 110. Accordingly, the alert generator 140 and the alert selection subsystem 116 may be housed in a common device. In some implementations, the alert generator 140 is housed in a first device, and the alert selection subsystem 116 is housed in a second device different from the first device.

In some implementations, based on the BG levels satisfying a low blood sugar criterion—e.g., the BG levels drop below a threshold (e.g., less than 70 milligrams per deciliter (mg/dL)—the alert selection subsystem 116 selects a particular alert type. In some implementations, the alert selection subsystem 116 selects a first alert type for a first sleepiness score and a selects a second alert type for a second sleepiness score. For example, for a relatively low sleepiness score (e.g., low confidence that the individual 150 is asleep), the alert selection subsystem 116 selects an audio alert type, whereas for a relatively high sleepiness score (e.g., high confidence that the individual 150 is asleep), the alert selection subsystem 116 selects a haptic alert type. As another example, for a relatively low sleepiness score (e.g., low confidence that the individual 150 is asleep), the alert selection subsystem 116 selects an audio alert type of a first intensity (e.g., first volume), whereas for a relatively high sleepiness score (e.g., high confidence that the individual 150 is asleep), the alert selection subsystem 116 selects an audio alert type of a second intensity (e.g., second volume) higher than the first intensity.

In some implementations, the alert generator 140 generates, based on the particular alert type (selected by the alert selection subsystem 116), an output that is directed to the individual 150. To that end, in some implementations, the alert generator 140 includes an audio output device (e.g., a speaker) that plays audio according to the particular alert type. For example, the volume (e.g., intensity) of the audio is indicated by the particular alert type. To that end, in some implementations, the alert generator 140 includes a haptic output device (e.g., a vibrating device) that generates a vibration. The haptic output device may be adapted to fit on (e.g., be affixed to) the individual 150. For example, the intensity of the vibration may be indicated by the particular alert type. In some implementations, the alert generator 140 includes multiple alerting output devices, such as two or more of an audio output device, an haptic output device, a temperature output device (e.g., heating element), etc.

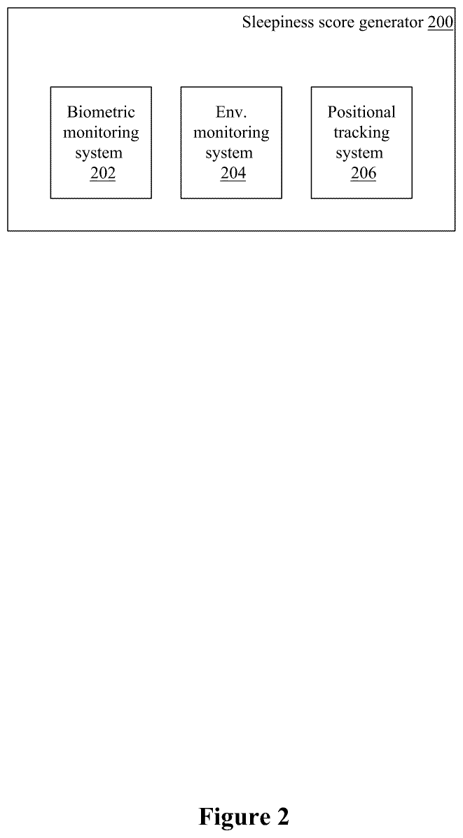

FIG. 2 is an example of a block diagram of a sleepiness score generator 200 in accordance with some implementations. In some implementations, the sleepiness score generator 200 is similar to and adapted from the sleepiness score generator 100 described with reference to FIG. 1. For example, in some implementations, the sleepiness score generator 200 is integrated in a smartwatch worn by an individual. As another example, the sleepiness score generator 200 is integrated in the BG system 110 of FIG. 1.

The sleepiness score generator 200 generates one or more sleepiness scores associated with an individual, as described with reference to the sleepiness score generator 130 of FIG. 1. For example, a sleepiness score indicates a sleeping status of the individual, such as a confidence the individual is asleep, a sleep stage of a sleeping individual, or a combination thereof.

According to various implementations, the sleepiness score generator 200 generates a sleepiness score based on one or more of an individual characteristic associated with an individual or an environmental characteristic associated with an environment of the individual. To that end, in various implementations, the sleepiness score generator 200 includes one or more of a biometric monitoring system 202, an environmental monitoring system 204, and a positional tracking system 206.

In some implementations, the individual characteristic associated with the individual includes one or more biometric values characterizing the individual. To that end, the sleepiness score generator 200 may include a biometric monitoring system 202 that generates biometric data indicating the biometric value(s). The biometric value(s) may include heart rate (e.g., beats per minute (BPM)), breathing rate, blood oxygen levels, etc. To that end, in some implementations, the biometric monitoring system 202 includes one or more of a heart rate sensor, a breathing rate sensor, a blood oxygen level sensor, etc., each of which generates corresponding biometric data. In some implementations, the biometric monitoring system 202 generates a plurality of biometric values associated with a particular biometric type, across a corresponding plurality of times. For example, the biometric monitoring system 202 generates a first biometric value indicating 70 BPM at a first time, and generates a second biometric value indicating 68 BPM at a second time later than the first time. Based on the biometric value(s), the sleepiness score generator 200 may generate a corresponding sleepiness score. For example, based on biometric values indicating a relatively low heart rate that has been steady for at least a threshold amount of time, the sleepiness score generator 200 determines a relatively high likelihood that the individual is sleeping and thus generates a relatively high sleepiness score. The sleepiness score generator 200 may use multiple biometric types to generate a sleepiness score. Continuing with the previous example, in addition to the biometric values associated with heart rate, the sleepiness score generator 200 obtains biometric values associated with breathing rate of the individual across a plurality of times. Because the biometric values associated with the breathing rate indicate a relatively low breathing rate that has been steady for at least the threshold amount of time, the sleepiness score generator 200 increases the sleepiness score because the sleepiness score generator 200 is even more confident that the individual is asleep, as compared with using only the biometric values associated with heart rate.

In some implementations, the individual characteristic associated with the individual corresponds to positional information regarding the individual. To that end, in some implementations, the sleepiness score generator 200 may include a positional tracking system 206 that generates positional data regarding the individual. For example, the positional tracking system 206 may include an inertial measurement unit (IMU) that generates positional data or movement data regarding the individual, such as an IMU integrated in a smartwatch worn by the individual or in a smartphone of the individual. Based on the positional data, the sleepiness score generator 200 may generate a sleepiness score. For example, based on the positional data or the movement data indicating less than a threshold amount of movement for at least a threshold amount of time, the sleepiness score generator 200 determines that the individual is likely asleep and thus generates a relatively high sleepiness score to reflect the high confidence that the individual is asleep. In contrast, based on the positional data or the movement data indicating relatively high levels of movement (e.g., the individual is exercising), the sleepiness score generator 200 generates a relatively low sleepiness score.

In some implementations, the environmental characteristic is associated with an environment of an individual. For example, an individual is currently inside a bedroom, and the environmental characteristic is associated with the bedroom. In some implementations, the environmental characteristic characterizes a physical feature of the environment. To that end, the sleepiness score may generator 200 may include an environmental monitoring system 204 that generates the environmental characteristic. For example, the environmental characteristic includes ambient light level of the environment. To that end, the environmental monitoring system 204 may include an ambient light sensor that generates ambient light sensor data regarding the environment. For example, based on the ambient light sensor data, the sleepiness score may generator 200 generates a higher sleepiness score for a darker environment (e.g., less ambient light) than for a lighter environment (e.g., more ambient light). As another example, in some implementations, the environmental monitoring system 204 includes an image sensor that captures image data of the environment, wherein the image data represents a sequence of images of the environment. In some implementations, the environmental monitoring system 204 assesses the image data to determine pixel values of pixels of the sequence of images. For example, based on detecting the majority of pixel values of pixels of an image are relatively dark (e.g., black pixel values or dark gray pixel values), the environmental monitoring system 204 determines that the individual is likely in a dark environment. Thus, the sleepiness score may generator 200 generates a higher sleepiness score than had the pixel values been of a lighter color. In some implementations, the environmental monitoring system 204 performs computer vision on the image data to generate one or more semantic values associated with the environment. For example, the environmental monitoring system 204 performs per-pixel semantic segmentation to generate a semantic value of “bed” associated with a physical bed in a bedroom. Because the semantic value of “bed” is typically associated with sleeping, the sleepiness score may generator 200 generates a higher sleepiness score than had the semantic values been of “trees” associated with physical trees in a forest, because trees are not associated with sleeping.

In some implementations, the sleepiness score may generator 200 uses data from two or more of the biometric monitoring system 202, the environmental monitoring system 204, and the positional tracking system 206 to generate a sleepiness score.

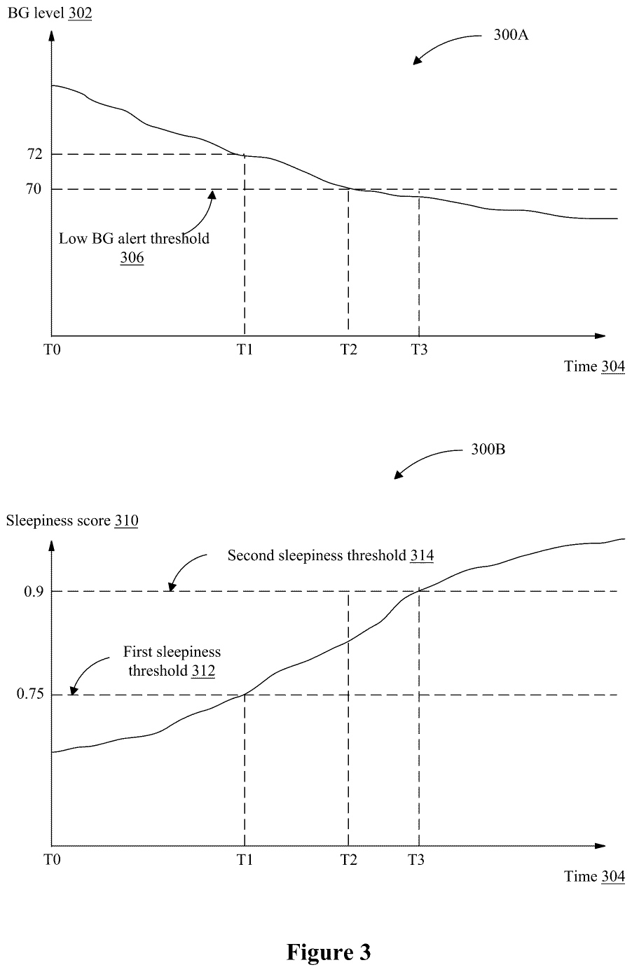

FIG. 3 is an example of a first timing diagram 300A and a second timing diagram 300B in accordance with some implementations.

The first timing diagram 300A is a graphical representation of BG level 302 (in (mg/dL) of an individual, changing over time 304. For example, the BG level 302 is indicated by BG data generated by a BG monitor, such as the BG monitor 120 described with reference to FIG. 1. For example, the BG data is generated by a CGM device that is worn by the individual. The first timing diagram 300A includes a low BG alert threshold 306 having a value of 70. Accordingly, in some implementations, when the BG level 302 of the individual drops below 70, the individual is alerted of the low BG level. One of ordinary skill in the art will appreciate that the low BG alert threshold 306 may have a value other than 70. One of ordinary skill in the art will further appreciate that some implementations include monitoring the BG level 302 to determine the BG level 302 remains below 70 for at least a threshold amount of time before alerting the individual. As illustrated in the first timing diagram 300A, the BG level 302 crosses (e.g., drops below) the low BG alert threshold 306 at a time T2. As further illustrated in the first timing diagram 300A, the BG level 302 remains below the low BG alert threshold 306 from T2 onwards.

The second timing diagram 300B is a graphical representation of a sleepiness score 310, changing over the time 304. The sleepiness score 310 indicates a confidence that the individual is asleep, and ranges from 0.0 (lowest confidence that the individual is asleep) to 1.0 (highest confidence that the individual is asleep). In some implementations, in addition to or instead of indicating a confidence that the individual is asleep, the sleepiness score 310 indicates a particular stage of sleep (e.g., REM versus NREM) that the individual is currently experiencing. In some implementations, the sleepiness score 310 is generated by a sleeping score generator, such as the sleeping score generator 200 described with reference to FIG. 2. The second timing diagram 300B includes a first sleepiness threshold 312 having a value of 0.75 and a second sleepiness threshold 314 having a value of 0.9. As illustrated in the second timing diagram 300B, the sleepiness score 310 crosses the first sleepiness threshold 312 at a time T1, which is before the time T2 when the BG level 302 crosses the low BG alert threshold 306. Moreover, the sleepiness score 310 crosses the second sleepiness threshold 314 at a time T3, which is after the time T2 when the BG level 302 crosses the low BG alert threshold 306.

According to various implementations and with reference to FIG. 1, the alert section subsystem 116 selects a particular alert type and directs the alert generator 140 to generate an alert output of the particular alert type, based on the crossing of one or more of the low BG alert threshold 306, the first sleepiness threshold 312, and the second sleepiness threshold 314. For example, in some implementations, the alert section subsystem 116 foregoes directing the alert generator 140 to generate an alert output before the time T2, because the BG level 302 does not cross the low BG alert threshold 306 before the time T2. As another example, in some implementations, the alert section subsystem 116 alerts the alert generator 140 to generate an alert output at the time T1, because the sleepiness score 310 crosses the first sleepiness threshold 312 at the time T1 and because at the time T1 the BG level 302 is above the low BG alert threshold 306 by an amount that is less than a threshold. For example, at the time T1 the BG level 302 is 72, which is less than 5% above the low BG alert threshold 306 of 70. Accordingly, in some implementations, the individual may be alerted of an imminent low BG level as the individual is on the verge of falling asleep (e.g., the individual is beginning to enter an early NREM sleep stage). This is helpful is preventing a hypoglemcic event, because an alert provided to the individual during an early sleep stage (rather than deep sleep) is more likely to wake the individual, enabling the individual to ingest glucose to avoid the hypoglemcic event.

In some implementations, because the low BG alert threshold 306 is crossed at the time T2, at the time T2 the alert section subsystem 116 selects a first alert type based on the sleepiness score having crossed the first sleepiness threshold 312 but not the second sleepiness threshold 314. For example, the alert section subsystem 116 selects an audio alert type, and directs the alert generator 140 to generate an audio output of the alert audio type. Moreover, at the time T3 the alert section subsystem 116 selects a second alert type (different from the first alert type) based on the sleepiness score having crossed the second sleepiness threshold 314. For example, the alert section subsystem 116 selects a haptic alert type, and directs the alert generator 140 to generate a haptic output of the haptic audio type. As another example, in some implementations, at the time T2 the alert section subsystem 116 directs the alert generator 140 to generate an alert type of a first intensity, and at the time T3 the alert section subsystem 116 directs the alert generator 140 to generate the alert type of a second intensity. The second intensity may be greater than the first intensity because the higher sleepiness score 310 at the time T3 (compared to at the time T2) indicates the individual is more likely to be sleeping and thus may need a more intense alerting mechanism to be awoken.

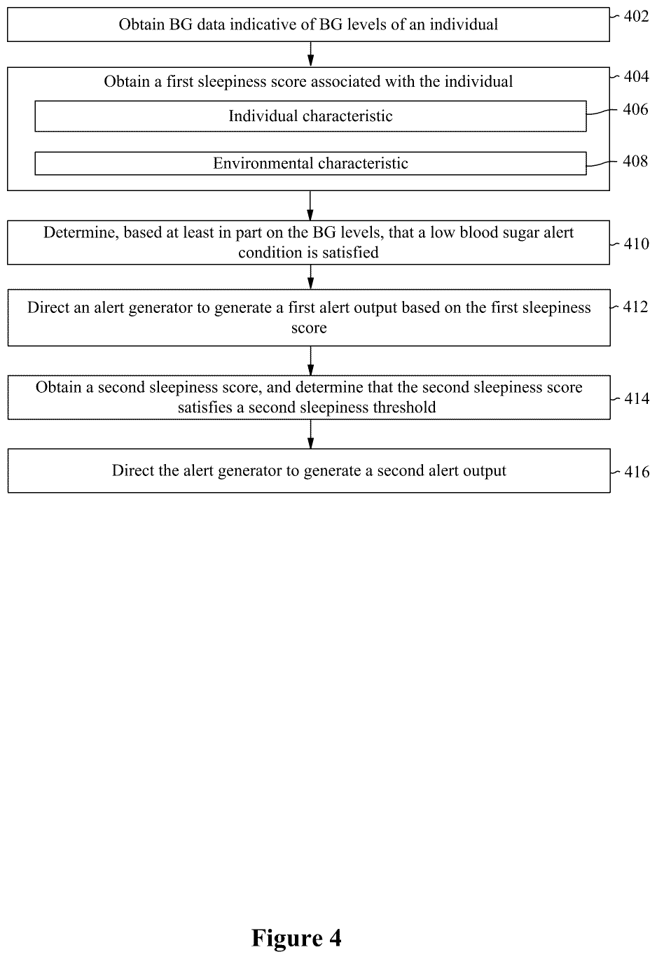

FIG. 4 is an example of a flow diagram of a method 400 of providing customized alerts according to various implementations. In various implementations, the method 400 or portions thereof are performed by a BG system, such as the BG system 110 described with reference to FIG. 1. In some implementations, the BG system is integrated in a CGM device or an insulin pump device. In various implementations, the method 400 or portions thereof are performed by an electronic device including one or more processors and a non-transitory memory, such as a CGM device, an insulin pump device, or a combination thereof. In some implementations, the method 400 is performed by processing logic, including hardware, firmware, software, or a combination thereof. In some implementations, the method 400 is performed by a processor executing code stored in a non-transitory computer-readable medium (e.g., a memory). In various implementations, some operations in method 400 are, optionally, combined and/or the order of some operations is, optionally, changed.

As represented by block 402, the method 400 includes obtaining BG data indicative of BG levels of an individual—e.g., the individual 150 of FIG. 1. For example, with reference to FIG. 1, the BG monitor 120 generates the BG data. As one example, the BG data is generated by a CGM device that is worn by (e.g., attached to skin of) the individual.

As represented by block 404, the method 400 includes obtaining a first sleepiness score associated with the individual. For example, the sleepiness score generator 130 described with reference to FIG. 1 generates the first sleepiness score. As another example, the sleepiness score generator 200 described with reference to FIG. 2 generates the first sleepiness score. The first sleepiness score may indicate a current sleep status of the individual. For example, the first sleepiness score indicates a confidence the individual is asleep, a current sleep stage of the individual, or a combination thereof. In some implementations, the first sleepiness score is independent of current time of day. Accordingly, the method 400 accounts for situations where the individual is sleeping during the day (e.g., at noon), in contrast to other techniques which may assume nighttime is when sleep occurs.

As represented by block 406, in some implementations, the first sleepiness score is based on an individual characteristic associated with the individual. For example, the individual characteristic corresponds to a biometric value indicated in biometric data associated with the individual. As one example, with reference to FIG. 2, the biometric monitoring system generates the biometric data. Examples of the biometric value are heart rate, breathing rate, blood oxygen levels, etc. As one example, in some implementations, the method 400 includes determining the first sleepiness score by assessing the steadiness or level of the heart rate over time. Continuing with this example, the method 400 may include assigning a relatively high value to the first sleepiness score (e.g., high confidence the individual is sleeping) based on a relatively low average heart rate (e.g., 60 BPM) that has been substantially constant (e.g., less than 5% deviation) over a certain period of time. In some implementations, the individual characteristic corresponds to positional information regarding the individual, which, for example, is generated by the positional tracking system 206 of FIG. 2. For example, the individual is wearing a smartwatch that includes an IMU, which generates positional or movement data. As one example, based on the positional information indicating that the individual has remained substantially stationary for at least a threshold amount of time, the method 400 may include assigning a relatively high value to the first sleepiness score (e.g., high confidence the individual is sleeping).

As represented by block 408, in some implementations, the first sleepiness score is based on an environmental characteristic associated with an environment of the individual. For example, the individual is physical located with the environment. In some implementations, the environmental characteristic is based on ambient light sensor data from an ambient light sensor, and the method 400 includes determining the first sleepiness score based on the ambient light sensor data. For example, the first sleepiness score may be inversely proportional to the amount of ambient light (e.g., the luminance), because the individual is more likely to be sleeping when there is less ambient light (e.g., a darker bedroom). In some implementations, the environmental characteristic is based on image data regarding the environment, and the method 400 includes determining the first sleepiness score based on the image data. For example, the image data is captured via an image sensor (e.g., a camera) that is integrated in a device performing the method 400. The image data may represent a series of images of the environment. In some implementations, the method 400 includes performing computer vision on the image data to determine the first sleepiness score. For example, pixel values of pixels of the image data are assesses to determine whether the environment is a dark or light, wherein a darker environment may result in a higher first sleepiness score. As another example, the method 400 may include performing computer vision (e.g., semantic segmentation) with respect to the image data to generate one or more semantic values associated with the environment. For example, a semantic value of “bed” may result in a higher first sleepiness score than a semantic value of “tree.”

As represented by block 410, the method 400 includes determining, based at least in part on the BG levels, that a low blood sugar alert condition is satisfied. For example, in some implementations, determining the low blood sugar alert condition is satisfied includes determining a current BG level, of the BG levels, crosses below a low BG alert threshold, such as the time T2 (illustrated in FIG. 4) when the BG level 302 crosses the low BG alert threshold 306. In some implementations, a CGM device includes a display with a user interface that enables an individual to manually set the low BG alert threshold—e.g., via a touch screen input to the display. The display may include real time BG levels, such as a graph that shows how BG levels change over time—e.g., display a line graph of BG levels over the past three hours.

In some implementations, determining the low blood sugar alert condition is satisfied is also based on the first sleepiness score associated, thereby enabling alerting the individual before the individual has reached a low BG level. For example, determining the low blood sugar alert condition is satisfied includes determining that a current BG level, of the BG levels, is above a low BG alert threshold by an amount that is less than a threshold, and determining the first sleepiness score crosses a sleepiness threshold. As one example, with reference to FIG. 4, at the time T1 the BG level 302 of 72 has not yet crosses the low BG alert threshold 306 of 70, but at the time T1 the sleepiness score 310 crosses the first sleepiness threshold 312. Continuing with this example, in some implementations, the method 400 includes determining that the BG level 302 of 72 at the time T1 is sufficiently close to the BG alert threshold 306 of 70 (e.g., less than 5% above 70). Accordingly, in some implementations, the method 400 includes proactively alerting an individual who is close to having a low BG level and about to fall asleep or already sleeping.

As represented by block 412, the method 400 includes, in response to determining the low blood sugar alert condition is satisfied, directing an alert generator to generate a first alert output based on the first sleepiness score. For example, directing the alert generator to generate the first alert output includes determining that the first sleepiness score crosses a first sleepiness threshold, such as the sleepiness score 310 crossing the first sleepiness threshold 312 of 0.75 at time T1 illustrated in FIG. 3.

In some implementations directing the alert generator to generate the first alert output includes selecting an alert type of a plurality of alert types based on the first sleepiness score, and transmitting, to the alert generator, an indication of the alert type. To that end, in some implementations, directing the alert generator includes transmitting, to the alert generator, data indicative of the first alert output, such as audio data (for an audio alert type) or haptic data (for a haptic alert type). In some implementations, selecting the alert type includes is based on a plurality of sleepiness thresholds. The plurality of alert types may include an audio alert type, a haptic alert type, a temperature alert type, etc. For example, referring back to FIG. 3, the method 400 includes selecting an audio alert type for the first alert output based on the sleepiness score 310 crossing the first sleepiness threshold 312 of 0.75 at time T1, and selecting a haptic alert type for a second alert output based on the sleepiness score 310 crossing the second sleepiness threshold 314 of 0.9 at time T3. In some implementations, the first sleepiness threshold is associated with a first sleep stage, and wherein the second sleepiness threshold is associated with a second sleep stage different from the first sleep stage. For example, the first sleepiness threshold is associated with the NREM sleep stage, and the second sleepiness threshold is associated with the REM sleep stage.

In some implementations, the alert generator is physically separate from the BG system. For example, the BG system is integrated in a first device, and the alert generator is integrated in a second device that is different from the first device. As one example, the BG system is integrated in a CGM device worn by the individual, and the alert generator is integrated in a smartwatch device worn by the individual. In some implementations, the alert generator is communicatively coupled (e.g., via Bluetooth or Wi-Fi) with the BG system to enable the BG system to direct the alert generator. To that end, in some implementations, the BG system includes a communication interface to communicate with the alert generator, and the BG system transmits, via the communication interface, the indication of the selected alert type to the alert generator.

In some implementations, the BG system and the alert generator are integrated in a common (e.g., the same) device. For example, each of the BG system and the alert generator is integrated in a CGM device.

In some implementations, the method 400 includes generating the first alert output. For example, in some implementations, in response to determining the low blood sugar alert condition is satisfied, the method 400 includes generating, via a haptic output device, a haptic output that is based on the first sleepiness score. For example, the intensity of the haptic output is proportional to the first sleepiness score—e.g., a higher confidence that the individual is sleeping result in a corresponding high vibration intensity, in order to increase the likelihood of waking the individual.

As represented by block 414, in some implementations, the method 400 includes, obtaining a second sleepiness score associated with the individual, and determining that the second sleepiness score satisfies a second sleepiness threshold. The second sleepiness threshold is different from the first sleepiness threshold, which is described with reference to block 412. Moreover, as represented by block 416, in some implementations the method 400 may include directing the alert generator to generate a second alert output in response to determining that the second sleepiness score satisfies the second sleepiness threshold. The second alert output is different from the first alert output, which is described with reference to block 412. In some implementations, the first alert output has a first alert characteristic, and the second alert output has a second alert characteristic different from the first alert characteristic. For example, the first alert output is of a first alert type, and the second alert output is of a second alert type different from the first alert type. As one example, with reference to FIG. 3, the method 400 includes directing the alert generator to generate the first alert output occurs based on determining the sleepiness score 310 crosses the first sleepiness threshold 312 at the time T1, and directing the alert generator to generate the second alert output occurs based on determining the sleepiness score 310 crosses the second sleepiness threshold 314 at the time T2. Continuing with this example, the first alert output is of an audio type and the second alert output is of a haptic audio type, of vice versa. As another example, the first alert output and the second alert output are of a common alert type, but with differing intensities, such as different volumes for an audio alert type or different vibration strengths for a haptic alert type.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or systems stored in a memory or other non-transitory computer-readable storage medium or device. The various functions disclosed herein may be implemented in such program instructions, although some or all of the disclosed functions may alternatively be implemented in application-specific circuitry (e.g., ASICs or FPGAs or GP-GPUs) of the computer system. Where the computer system includes multiple computing devices, these devices may be co-located or not co-located. The results of the disclosed methods and tasks may be persistently stored by transforming physical storage devices, such as solid-state memory chips and/or magnetic disks, into a different state.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various implementations described above can be combined to provide further implementations. Accordingly, the novel methods and systems described herein may be implemented in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. A method comprising:
obtaining blood glucose (BG) data indicative of BG levels of an individual;
obtaining a first sleepiness score associated with the individual;
determining, based at least in part on the BG levels, that a low blood sugar alert condition is satisfied; and
in response to determining the low blood sugar alert condition is satisfied, directing an alert generator to generate a first alert output based on the first sleepiness score.

2. The method of claim 1, wherein determining the low blood sugar alert condition is satisfied includes determining that a current BG level, of the BG levels, crosses below a low BG alert threshold.

3. The method of claim 1, wherein determining the low blood sugar alert condition is satisfied is also based on the first sleepiness score.

4. The method of claim 3, wherein determining the low blood sugar alert condition is satisfied includes:
determining that a current BG level, of the BG levels, is above a low BG alert threshold by an amount that is less than a threshold; and
determining that the first sleepiness score crosses a sleepiness threshold.

5. The method of claim 1, wherein the first sleepiness score is based on one or more of an individual characteristic associated with the individual or an environmental characteristic associated with an environment of the individual.

6. The method of claim 5, wherein the first sleepiness score is based on the individual characteristic and the environmental characteristic.

7. The method of claim 5, wherein the individual characteristic corresponds to a biometric value indicated in biometric data associated with the individual.

8. The method of claim 5, wherein the individual characteristic corresponds to positional information regarding the individual.

9. The method of claim 5, wherein the environmental characteristic is based on image data regarding the environment.

10. The method of claim 9, wherein determining the first sleepiness score includes performing computer vision with respect to the image data to generate one or more semantic values associated with the environment.

11. The method of claim 5, wherein the environmental characteristic is based on ambient light sensor data from an ambient light sensor, and wherein determining the first sleepiness score is based on the ambient light sensor data.

12. The method of claim 1, wherein the first sleepiness score is independent of current time of day.

13. The method of claim 1, wherein directing the alert generator to generate the first alert output includes:
selecting an alert type of a plurality of alert types based on the first sleepiness score; and
transmitting, to the alert generator, an indication of the alert type.

14. The method of claim 13, wherein the alert generator includes an output device, the method further comprising generating, via the output device, the first alert output of the alert type based on the indication.

15. The method of claim 14, wherein the alert type corresponds to an audio alert type, wherein the output device corresponds to an audio output device, and wherein generating the first alert output includes playing a tone via the audio output device.

16. The method of claim 14, wherein the alert type corresponds to a haptic alert type, wherein the output device corresponds to a haptic output device, and wherein generating the first alert output includes generating a haptic output via the haptic output device.

17. The method of claim 1, wherein directing the alert generator to generate the first alert output includes determining that the first sleepiness score crosses a first sleepiness threshold.

18. The method of claim 17, further comprising:
obtaining a second sleepiness score associated with the individual;
determining that the second sleepiness score satisfies a second sleepiness threshold different from the first sleepiness threshold; and
directing the alert generator to generate a second alert output in response to determining that the second sleepiness score satisfies the second sleepiness threshold, wherein the second alert output is different from the first alert output.

19. The method of claim 18, wherein the first alert output has a first alert characteristic, and wherein the second alert output has a second alert characteristic different from the first alert characteristic.

20. The method of claim 19, wherein the first alert characteristic corresponds to a first alert type, and wherein the second alert characteristic corresponds to a second alert type different from the first alert type.

21. The method of claim 20, wherein the first alert type corresponds to a haptic alert, and wherein the second alert type corresponds to an audio alert.

22. The method of claim 19, wherein each of the first and second alert characteristics is of a common alert type, wherein the first alert characteristic is of a first intensity level, and wherein the second alert characteristic is of a second intensity level that is different from the first intensity level.

23. The method of claim 18, wherein the first sleepiness threshold is associated with a first sleep stage, and wherein the second sleepiness threshold is associated with a second sleep stage different from the first sleep stage.

24. The method of claim 23, wherein the first alert output is associated with a first intensity level, and wherein the second alert output is associated with a second intensity level that is higher than the first intensity level.

25. A blood glucose (BG) system comprising:
a BG retrieval subsystem to obtain BG data indicative of BG levels of an individual;
a sleepiness score retrieval subsystem to obtain a first sleepiness score associated with the individual; and
an alert selection subsystem to:
determine, based at least in part on the BG levels, that a low blood sugar alert condition is satisfied; and
direct an alert generator to generate a first alert output based on the first sleepiness score, in response to determining that the low blood sugar alert condition is satisfied.

26. The BG system of claim 25, wherein the BG system is integrated in a first device, and wherein the alert generator is integrated in a second device that is different from the first device.

27. The BG system of claim 26, wherein the first device includes a communication interface to communicate with the second device, and wherein the alert selection subsystem transmits, via the communication interface, an alert instruction to the second device to direct the alert generator to generate the first alert output.

28. The BG system of claim 25, wherein the BG system and the alert generator are integrated in a common device.

29. The BG system of claim 25, wherein the BG system includes the alert generator, and wherein the alert generator generates the first alert output based on the direction.

30. The BG system of claim 25, wherein the BG system is integrated in a continuous glucose monitor (CGM) device.

31. The BG system of claim 25, wherein the BG system is integrated in an insulin pump device.

32. (canceled)

33. A non-transitory computer-readable medium that includes instructions stored thereon, which, when executed by one or more processors, cause the one or more processors to perform operations comprising:
obtaining blood glucose (BG) data indicative of BG levels of an individual;
obtaining a first sleepiness score associated with the individual;
determining, based at least in part on the BG levels, that a low blood sugar alert condition is satisfied; and
in response to determining the low blood sugar alert condition is satisfied, directing an alert generator to generate a first alert output based on the first sleepiness score.
